(19) United States
(12) Patent Application Publication
(10) Pub. No.: US 2005/0019422 A1
(43) Pub. Date: Jan. 27, 2005

(54) Method and composition for treating acne

(75) Inventor: Brent A. Johnson, Rancho Santa Margarita, CA (US)

Correspondence Address: BRENT A. JOHNSON;ALLERGAN, INC., 2525 Dupont Drive, T2-7H, Irvine, CA, 92612, US

(73) Assignee: Allergan, Inc.

(21) Appl. No.: 10/625,969

(22) Filed: Jul. 23, 2003

(51) Int. Cl. A61K 33/40; A61K 31/60; A61K 31/19

(52) U.S. Cl. 424/616; 514/159; 514/558; 514/568

(57) ABSTRACT

This invention relates to compositions and methods for treating acne, wherein said compositions comprise two therapeutically active agents. One of the therapeutically active agents is a peroxide, and the other therapeutically active agent is a carboxylic acid, or a salt or an ester thereof. The two therapeutically active agents are excluded from contact with one another by entrapping one of the therapeutically active agents in a multiplicity of particles or beads.

### FIELD OF THE INVENTION

This invention relates to methods and compositions for treating acne vulgaris.

### BACKGROUND OF THE INVENTION

### DESCRIPTION OF THE RELATED ART

Acne vulgaris is an inflammatory disease of the sebaceous glands characterized by an eruption of the skin, often pustular in nature but not suppurative. Acne is a common affliction of the adolescent and affects a small but significant percentage of the adult population which results in unsightly lesions, particularly on the face, and in some cases severe scarring.

Various topical agents are utilized in the treatment of acne and these include sulfur, resorcinol, salicylic acid, benzoyl peroxide, retinoids and topical antibiotics. An effective anti-acne agent (or composition) must exhibit the following activities:
(a) a sebostatic activity so as to inhibit hyperseborrhea;
(b) a keratolytic and comedolytic activity so as to avoid hyperkeratosis of the follices and to permit removal of comedos;
(c) a bacteriostatic activity so as to inhibit the activity of Propionibacterium acnes.
   Nevertheless, acne vulgaris is seldom cured and only can be contained with difficulty.

Peroxides are commonly used for the treatment of acne. In particular, benzoyl peroxide has been suggested for treating acne vulgaris. (See U.S. Pat. No. 4,387,107.) For many years, benzoyl peroxide has been proven to be a particularly powerful keratolytic and anti-seborrhic agent, as well as being endowed with antibacterial properties. Topical benzoyl peroxide compositions, including a vehicle to enhance the efficacy thereof, are known (See U.S. Pat. No. 4,411,893). Topical compositions of benzoyl peroxide combination with antibiotics are also known. (See U.S. Pat. Nos. 4,407,794; 4,692,329 and 4,387,107)

Peroxides, other than benzoyl peroxide, have also been suggested for treatment of acne vulgaris, alone or in combination with other compounds useful in treating acne vulgaris. (See U.S. Pat. Nos. 4,607,101 and 4,906,617.) These peroxides are suggested as having certain advantages, e.g. stability over benzoyl peroxide. U.S. Pat. No. 4,671,956 identifies the problem of benzoyl peroxide decomposing coingredients in topical formulations to thereby cause itching upon application. It was suggested that this problem may be solved by including a sunscreen in the topical formulation to retard this decomposition effect of benzoyl peroxide.

Carboxylic acids, and their salts and esters, have been used for some time in the treatment of acne. One particularly useful carboxylic acid in the treatment of acne is salicylic acid, which is present in a number of readily available acne treatments. Certain retinoid compounds which are carboxylic acids, or salts or esters thereof, are also known to be useful in treating acne. For example, tazarotene, which is marketed by Allergan, Inc. under the trade name Tazorac®, is sold for the topical treatment of acne. Tazarotene is an ethyl ester of a carboxylic acid. Another carboxylic acid, azelaic acid has been used topically and systemically to treat acne (U.S. Pat. No. 4,386,104 to Nazzo-Pavarro), and topical compositions including benzoyl peroxide and azelaic acid for the treatment of acne have been disclosed in the commonly owned U.S. Pat. No. 6,262,117.

Particulate encapsulation is a commonly used technique in cosmetic and related arts. In this technique, incompatible components of a composition are effectively separated by encapsulating one of the incompatible components in multiplicity of beads or particles. A number of patents have been issued related to the use and preparation of these beads or particles, including U.S. Pat. No. 6,413,548 and U.S. Pat. No. 5,598,194, incorporated herein by reference. U.S. Patent Application Publication No. U.S. 2002/006451 A1 and WO 018023 A2 disclose the use of microcapsules comprising at least one inorganic polymer obtained by a sol-gel process, in stabilizing reactive ingredients of a formulation, including benzoyl peroxide.

Despite the known incompatibilities of benzoyl peroxide with other formulation ingredients, none of the references disclosed herein suggest any need to prevent contact between a peroxide and a carboxylic acid, or an ester or salt thereof, in a composition useful for the treatment of acne. In fact, U.S. Pat. No. 4,671,956 teaches the opposite, in disclosing that esters of salicylate and cinnamate are useful in stabilizing benzoyl peroxide in compositions for treating acne. In making the above statements, the applicant makes no admission as to whether any of the references cited herein are prior art.

### SUMMARY OF THE INVENTION

It is therefore surprising that topical compositions will be unstable in situations where peroxides and carboxylic acids, or salts or esters thereof, come into direct contact with each other. While not intending to be bound in any way by theory, it is believed that the peroxide will oxidize the carboxylic acid to a peroxy acid, in a manner similar to that disclosed by March (Advanced Organic Chemistry Reactions, Mechanism, and Structure, Fourth Edition, New York: John Wiley and Sons, 1992, p. 1203). This oxidation of the carboxylic acid thus destabilizes both active ingredients, having an adverse effect upon the shelf life of the product.

One aspect of this invention relates to a heterogeneous pharmaceutical composition which is useful for the treatment of acne comprising two different therapeutically active agents in effective amounts. One of these therapeutically active agents comprises a carboxylic acid, or a salt or ester thereof. The other therapeutically active agent comprises a peroxide. In relation to this invention, a multiplicity of solid particles comprises one therapeutically active agent such that the other therapeutically active agent is effectively excluded from said particles. Thus, by reducing the contact between the two incompatible therapeutically active agents in the composition, they are stabilized such that the shelf life of the composition is improved.

Another aspect of this invention relates to methods of preparing a pharmaceutical composition comprising a peroxide and a carboxylic acid, or a salt or an ester thereof.

Another aspect of this invention relates to methods of stabilizing a carboxylic acid, or a salt or an ester thereof, in a pharmaceutical product also comprising a peroxide.

Another aspect of this invention relates to pharmaceutical products comprising water, a multiplicity of solid particles, two therapeutically active agents, and a package for the dispensing of the therapeutically active agents.

### DETAILED DESCRIPTION OF THE INVENTION

In addition to relating to compositions of matter, this invention also relates to a pharmaceutical product comprising water, a multiplicity of solid particles, two therapeutically active agents, and a package. In this aspect of the invention the first therapeutically active agent is a carboxylic acid, or a salt or an ester thereof, and the second therapeutically active agent is a peroxide. In this aspect of the invention, one of the therapeutically active agents is entrapped in said multiplicity of solid particles such that contact between the therapeutically active agents is substantially reduced. Additionally, the package is suitable for dispensing the therapeutically active agents and the product is indicated for the treatment of acne.

Another aspect of this invention relates to a method of preparing a pharmaceutical composition. This method comprises the following three steps, not necessarily in the order listed:
a. providing two therapeutically active agents in effective amounts, one therapeutically active agent being a carboxylic acid, or a salt or an ester thereof; and another therapeutically active agent being a peroxide;
b. providing a multiplicity of solid particles comprising one of the two therapeutically active agents in a manner that substantially excludes contact between the two therapeutically active agents;
c. providing an aqueous liquid wherein said solid particles are dispersed said aqueous liquid phase;
   wherein said composition is useful for the treatment of acne.

This invention also relates to a method of stabilizing a carboxylic acid, or a salt or ester thereof in a pharmaceutical product also comprising a peroxide, wherein said product is useful for the treatment of acne. This method comprises the following two steps, not necessarily in the order listed:
a. entrapping the carboxylic acid, or salt or ester thereof in a multiplicity of solid particles such that the peroxide is effectively excluded from said particles; or
b. entrapping the peroxide in a number of solid particles such that the carboxylic acid, or salt or ester thereof is effectively excluded from said particles.

In relation to this invention, the term heterogeneous pharmaceutical composition means that the composition is not homogeneous, meaning that it does not form a clear solution, but that the solid particles are dispersed within the liquid phase. Reference to the composition as a pharmaceutical composition indicates that the composition is used for the treatment or prevention of a disease or adverse condition afflicting a person.

The terms carboxylic acid, and salt or esters thereof have the broadest meaning commonly understood by organic chemists. While not intending to be limiting, a salt could be formed by inorganic cations having a charge of +1 or higher such as Na⁺, K⁺, Ca²⁺, Mg²⁺, and NH₄⁺, or could be formed by organic cations. In certain embodiments of this invention, the carboxylic acid, or salt or ester thereof, comprises a retinoid compound such as tazarotene. While not intending to be bound in any way by theory, this invention is also beneficial in embodiments comprising retinoids in that retinoids have sites of unsaturation, and in some cases, heteroatoms that are particularly susceptible to peroxides. For example, tazarotene has an alkynyl group and a thioether, both of which are susceptible to oxidation by a peroxide. In other embodiments the carboxylic acid, or salt or ester thereof comprises compounds which are not retinoids such as salicylic acid or azelaic acid, or as salt or ester based on one of these carboxylic acid.

A peroxide according to the invention herein is a compound comprising an O—O single bond, including, but not limited to, hydrogen peroxide; alkyl peroxides, where one oxygen atom is bonded to an alkyl group and the other oxygen atom is bonded to hydrogen; dialkyl peroxides, where each oxygen is bonded to an alkyl group, diacyl peroxides; where each oxygen is bonded to an acyl group; and peroxy acids where one oxygen is bonded to an acyl group, and the second oxygen is bonded to a hydrogen. In certain embodiments of this invention, the peroxide is hydrogen peroxide. In other embodiments, the peroxide is benzoyl peroxide.

In relation to the present invention, the phrase multiplicity of solid particles refers to any number of solid particles which comprise one of the therapeutically active agents. In relation to the particles and one of the therapeutically active agents, the term entrapping refers to incorporating the therapeutically active agent into the structure of the solid particles. In relation to this invention, the terms particles and beads may be used interchangeably. While not intending to limit the scope of the invention in any way, the therapeutically active agent is generally dissolved or otherwise dispersed in the solid particle, depending on the particular properties of the therapeutically active agent and the other components of the solid particles. For example, if the particles are prepared by cooling a liquid, the therapeutically active agent may be soluble in the liquid, and the solution may freeze into a solid solution. In other cases, the therapeutically active agent is not soluble in liquid that is cooled to form the particle, in which case the particles may be a heterogeneous dispersion of a solid or liquid in a solid. Other types of particles are also contemplated, such as particles which comprise a core having the therapeutically active agent, where the core is coated by an inert material.

The multiplicity of particles is intended to effectively exclude or substantially exclude contact between the two incompatible therapeutically active ingredients. The terms effectively and substantially used in relation to the exclusion of contact are meant to account for the fact that incidental contact is likely to occur in many instances, such as at the phase boundary or interface between a particle and the remainder of the composition. It is also possible that a residual amount of free therapeutically active agent could be present on the surface of the particle and be released into the surrounding material upon mixing. There are also a number of ways in which small amounts of the two therapeutically active agents could have contact as a result of contamination or other aspects of the manufacturing process. A person skilled in the art will recognize that there are other ways that contact could be made between the two therapeutically active agents, while contact between them is still effectively or substantially excluded within the scope of the present invention.

In relation to this invention, either of the two therapeutically active agents could be comprised in the multiplicity of particles and either of the two therapeutically active agents could be comprised in the remainder of the composition, provided that contact between the two therapeutically active agents are effectively excluded from contact. However, while not intending to limit the scope of the invention in any way, there are considerations that one should make when practicing the invention. For example, peroxides tend to be unstable, and can explode when they become too concentrated. Therefore, care should be taken in a situation where the beads comprise a peroxide at a high concentration, particularly if the part of the preparation of the beads is carried out at relatively high temperatures. For this reason, in many cases it may be advantageous to incorporate the carboxylic acid, or salt or ester thereof, into the particles. However, incorporating the peroxide into the particles is advantageous in certain situations, such as when protecting certain excipients that are less stable in the presence of a peroxide is desirable, or in situations where the particles might be useful in helping to stabilize the peroxide, or for other reasons apparent to those of ordinary skill in the art depending on the circumstances. Thus, while not intending to limit the scope of the invention in any way, the individual circumstances in which the invention is practiced may provide guidance to one of ordinary skill in the art as to which therapeutically active agent is comprised by the particles in practicing the invention.

The therapeutically active agent not comprised by the particles could be either dissolved or dispersed in the aqueous phase. Thus, the compositions of this invention may comprise two phases-solid particles dispersed in an aqueous solution, or the compositions of this invention may comprise three or more phases, with the second therapeutically active agent and possibly one or more of the excipients, being dispersed as a solid or liquid in the aqueous phase. Other forms for the composition are also possible, such as those that comprise emulsions and can be readily practiced by those of ordinary skill in the art, and thus fall within the spirit and scope of this invention.

While not intending to limit the scope of the invention in any way, in certain situations it will be beneficial to incorporate a viscosity enhancing agent into the compositions of the present invention. This may help keep the particles dispersed during shipping and storage, or in other words, retard settling of the particles. Furthermore, in some situations increasing the viscosity of the compositions will facilitate the administration of the therapeutically active agents. While not intending to limit the scope of the invention in any way, the viscosity-enhancing agent may comprise a polymer containing hydrophilic groups such as monosaccharides, polysaccharides, ethylene oxide groups, hydroxyl groups, carboxylic acids or other charged functional groups. Some examples of viscosity-enhancing agents useful in the present invention are sodium carboxymethylcellulose, hydroxypropylmethylcellulose, povidone, polyvinyl alcohol, and polyethylene glycol.

The multiplicity of particles should effectively deliver the therapeutically active agent it comprises to the person or mammal upon topical administration of the product. This delivery could be effected in any number of ways. While not intending to be limiting, the particles could be broken by the friction of rubbing when the product is administered, or the particles might melt upon contact with the skin. While not intending to limit the scope of the invention in any way, in relation to the administration and dispensing of the composition of this invention, the properties of the multiplicity of particles of beads can be tuned to the particular circumstances. In this regard, the melting point of the particles is an important consideration. In many situations, it is useful for the melting point of the particles to be from 30° C. to 70° C. For example, in certain situations it is useful for the melting point of the beads to be around or just above the body temperature of a human being, or from 35° C. to 42° C. This would allow the particles to be broken or crushed more readily upon topical administration. In other situations it would be useful for the melting point of the beads to be around or just below the body temperature of a human being, or from 31° C. to 36° C., so they would melt readily upon contact with the skin, thus dispensing the therapeutically active agent. These considerations may be weighed against other factors important to the manufacture, packaging, shipping and storage of the product. For example, the manufacturing process may involve steps that have a temperature around or higher than human body temperature, in which case it might be preferable for the melting point of the beads to be higher than body temperature. In many cases, elevated temperatures may occur during shipping and storage, so depending upon the particular circumstances it may be useful for the melting point of the beads to be between about 35° C. to 50° C. Practical considerations related to the use of liquid water in the compositions of this invention may dictate that the melting point of the particles should be greater than 20° C. and less than 100° C. However, the melting temperatures of the particles described herein are given only to provide guidance as to how to make and use the invention, and are not intended to limit the overall scope of the invention in any way.

While not intending to limit the scope of the invention in any way, the size of the particles is another important consideration in relation to this invention. The particles should be small enough so that the therapeutically active agent that they comprise is effectively distributed throughout the composition and so that they are able to effectively be dispensed. Thus, the package from which the compositions of the present invention are dispensed may also be an important consideration in relation to the size of the particle. In terms of the packaging, it may be useful to have the composition dispensed from a container which has an opening having a diameter of greater than 1 cm. In many cases the diameter may also be 10 cm or less in diameter. This type of packaging allows the product to be dispensed by inserting a finger or other dispensing means such as a cotton swab through the opening so that the composition can be distributed upon the skin of the person the patient. This large opening also provides greater flexibility in terms of the size of the particles. In other situations, it is more convenient to dispense the product through a small opening, having a diameter of 0.5 mm to 1 cm, from a tube, bottle, or an equivalent type of packaging. In these instances, the particles should be sufficiently small to be effectively dispensed, in some cases having an average diameter from 0.1 mm to 1 mm. Additionally, all of the particles should be small enough to fit through the opening, which in many cases may mean that no particle has a diameter greater than 1 mm. Both in terms of the opening in the package from which the product is dispensed and the size of the particles, the term diameter should be understand broadly, meaning that it can apply to shapes that are not spherical, circular, cylindrical, etc. In terms of an opening in a package, the term diameter is defined as being the limiting (smallest) dimension in allowing an object to pass through it. In terms of a particle, the term diameter is defined as the limiting (largest) dimension in preventing the particle to pass through an opening. Thus, if the diameter of the particle is larger than the diameter of the opening in the package, it will not pass through the opening. It is often desirable that all of the particles be sufficiently small to pass through the opening. The considerations discussed here related to the size of the opening in the package and the solid particles are given merely for illustration and instruction purposes, and are not intended to limit the overall scope of the invention in any way.

The choice of materials comprised by the particles is an important consideration related to making and using this invention. Any material which can effectively exclude contact between the two therapeutically active agents, and can deliver the therapeutically active agent the particles comprise at the appropriate time is within the scope of this invention. However, while not intending to limit the scope of the invention, some useful materials for the beads are those that comprise solid fatty acid esters, both natural such as beeswax, Jojoba oil and coconut oil, and derived such as esters of stearic acid; petroleum based materials such as paraffin wax; and polymers.

While not intending to limit the scope of the invention in any way, the melting point of the particles can be adjusted by a person skilled in the art by the choice of materials, and by blending or chemical derivatization of those materials. Those of ordinary skill in the art know a number of chemical methods, such as transesterification, to increase of decrease the melting point of a composition. Alternatively, the melting point of the particles can be adjusted without chemical modification by blending one or more of the materials to form the base material. The term base material refers to all of the materials comprised in the particles which are not the therapeutically active agent. Melting points for some useful materials are listed in Table 1 below. A target melting point for the particles might be achieved, for example, by blending the therapeutically active agent with a base material that has a higher melting point than the target, and mixing in a lower melting point base material until the mixture has the desired melting point.

TABLE 1

| Material | Melting Point (° C.) |
|---|---|
| Beeswax (Yellow) | 62-65 |
| Paraffin wax | 50-57 |
| Methyl stearate | 40-42 |
| Coconut oil | 21-25 |
| Jojoba Oil | 6-10 |

As mentioned, preparation of the particles or beads is can be carried out by one of ordinary skill in the art without undue experimentation. However, while not intending to limit the scope of the invention in any way, these particles can be prepared by vigorously mixing the base materials with the therapeutically active agent above the melting point of the base materials, and possibly, the above the melting point of the therapeutically active agent, and forcing the material through a needle into a quenching material. The quenching material is generally at a temperature well below that of the material to be formed into particles. The material to be formed into particles is forced through the needle, and the stream of the hot mixture breaks up and freezes in the cold fluid to form small particles or beads. When all of the material has been frozen into the particles, the quenching material is evaporated or decanted. Alternatively, the beads are filtered, or isolation of the beads is carried out by some other means known to one of ordinary skill in the art. The average particle size and the size of the largest particle are readily modified by one of ordinary skill in the art. The average particle size, for example, could be changed by changing the inner diameter of the needle. The size of the largest particle is easily controlled by passing the beads through a sieve or some other means of filtering out large particles. The examples and instructions given herein regarding the preparation of the particles or beads are purely for purposes of illustration to help one to carry out the invention, and are not intended to limit the scope of the invention in any way. A person of ordinary skill in the art will recognize that there are many ways in which these particles or beads could be prepared, all of which fall within the spirit and scope of this invention.

In relation to the therapeutically active agents of the present invention, the term therapeutically effective amount refers to the amount of the agent required to achieve the desired result in terms of relieving the suffering of the individual being treated. While not intending to limit the scope of the invention in any way, it is often useful for the carboxylic acid, or the salt or ester thereof (e.g. azelaic acid, sodium salt or lower alkyl ester), to be present in concentrations of from 0.1 to 30 percent, and for the peroxide to be present in a concentration of from 0.1 to about 30 percent, in the compositions of this invention.

In addition to the considerations described above, it is often desirable that the compositions of the present invention include one or more pharmaceutically acceptable carriers that enhance the efficacy of topical administration. Pharmaceutically acceptable carriers include conventional emulsifiers, such as fatty alcohols, glycol ethers and esters of fatty acids; conventional emollients, such as isopropyl and butyl esters of fatty acids, e.g. isopropyl myristate; humectants such as glycerin, propylene glycol, polyethylene glycol; and alcohols and acetone; oils such as mineral oil, petroleum oil, oil extracts from animal or vegetable sources; conventional stabilizers including antioxidants and preservatives. The compositions may also include agents, such as urea, to improve the hydration of the skin. In addition to the foregoing conventional formulations, the topical compositions may include penetration-enhancing agents such as 1-pyrrolidone and N-lower alkyl-2-pyrrolidones, such as N-methyl-2-pyrrolidone; and 1-substituted azacycloalkan-2-ones such as, for example, 1-n-dodecylazacycloheptan-2-one and other compounds disclosed in U.S. Pat. No. 3,989,816. Longer chain sulfoxides, e.g., n-octyl methyl sulfoxide and hexamethylene-lauramide and the other penetration-enhancing agents disclosed in U.S. Pat. No. 4,743,588, may also be included in the formulations utilized in the method of this invention. The amount of the compositions to be administered will obviously be an effective amount for the desired result expected therefrom. This, of course, will be ascertained by the ordinary skill of the practitioner. In accordance with the usual prudent formulating practices, a dosage near the lower end of the useful range of the particular agent may be employed initially and the dosage increased as indicated from the observed response, as in the routine procedure of the physician.

In carrying out the novel method employing the topical route, the active ingredient(s) formulated, for example, as a gel or lotion or suspension, is applied to the affected area of the skin at a rate varying from 0.2 mg per square cm of skin surface per day up to 10 mg per square cm of skin surface per day until the appearance of the affected skin has returned to normal. The gel or lotion or suspension is generally applied for several days.

The topical compositions of this invention may be applied to the face of a patient with acne 1 to 4 times daily with the result that open and closed comedones are markedly reduced within two to four weeks.

### EXAMPLE 1

Azelaic acid (20 g) is ground to a fine powder and added to preheated beeswax (80 g) at 100° C. in a pressure vessel equipped with a heater, a mechanical agitator, a pressure source, and a needle valve on the bottom of the vessel with a 0.2 mm inner diameter needle attached. Jojoba Oil is then added in 5 g increments until the melting point of mixture is about 38-40° C. Azelaic acid is then added to adjust the composition of the mixture to 20% azelaic acid.

The tip of the needle is submerged below the surface of about 1700 mL of FC-77 perfluorocarbon fluid (3M, St. Paul, Minn.) in a tall 2 L beaker. The perfluorocarbon fluid is maintained at about 5° C. by an external cooling apparatus. The vessel is closed and stirred vigorously at 100° C. for 30 minutes. The needle valve is then opened, and sufficient pressure is applied to force a stream of the azelaic acid/beeswax mixture into the perfluorocarbon fluid. The stream of the hot mixture breaks up and freezes in the cold fluid to form small particles or beads having diameters around 0.1-1 mm. When all of the azelaic acid/beeswax mixture has been added and has solidified, the particles are filtered out and air dried.

The particles are then added to 100 g of Brevoxyl-8® (8% benzoyl peroxide gel, Stiefel Laboratories, Inc., Coral Gables, Fla. 33134 USA) and stirred to disperse the solid azelaic acid/beeswax particles.

### EXAMPLE 2

The composition of Example 1 is applied to the face of a patient with acne 1 to 4 times daily. Open and closed comedones are markedly reduced within two to four weeks.

1. A heterogeneous pharmaceutical composition comprising two different therapeutically active agents in effective amounts, said therapeutically active agents comprising:
a. a carboxylic acid, or a salt or ester thereof; and
b. a peroxide;
wherein a multiplicity of solid particles comprises one therapeutically active agent such that the other therapeutically active agent is effectively excluded from said particles and wherein said composition is useful for treating acne.

2. The composition of claim 1 wherein said carboxylic acid, or salt or ester thereof comprises azelaic acid, or a salt or ester thereof.

3. The composition of claim 1 wherein said carboxylic acid, or salt or ester thereof comprises salicylic acid, or a salt or ester thereof.

4. The composition of claim 1 wherein said carboxylic acid, or salt or ester thereof comprises azelaic acid.

5. The composition of claim 1 wherein said peroxide is hydrogen peroxide.

6. The composition of claim 1 wherein said peroxide is benzoyl peroxide.

7. The composition of claim 1 wherein said multiplicity of solid particles has an average particle diameter of from 0.1 mm to 1 mm.

8. The composition of claim 7 wherein no particle of said multiplicity of solid particles has a diameter larger than 1 mm.

9. The composition of claim 1 wherein said multiplicity of solid particles comprises a material selected from the group consisting of beeswax, Jojoba oil, coconut oil, methyl stearate, paraffin wax, and mixtures thereof.

10. The composition of claim 1 wherein the multiplicity of solid particles has a melting point of less than 100° C.

11. The composition of claim 1 wherein the multiplicity of solid particles has a melting point greater than 20° C.

12. The composition of claim 1 wherein the multiplicity of solid particles has a melting point from 30° C. to 70° C.

13. The composition of claim 1 wherein the multiplicity of solid particles has a melting point from 35° C. to 50° C.

14. The composition of claim 1 wherein the multiplicity of solid particles has a melting point from 35° C. to 42° C.

15. The composition of claim 1 wherein the multiplicity of solid particles comprises the carboxylic acid, or a salt or an ester thereof.

16. The composition of claim 1 wherein the multiplicity of solid particles comprises said peroxide.

17. The composition of claim 1 wherein the carboxylic acid is azelaic acid which is comprised in the multiplictiy of solid particles, said multiplicity of solid particles also comprising beeswax and Jojoba oil and having a melting point between 35-42° C.

18. The composition of claim 17 wherein said multiplicity of solid particles has an average particle diameter of 0.1 mm to 1 mm, and wherein no particle of said multiplicity of solid particles has a diameter larger than 1 mm.

19. A method of preparing a pharmaceutical composition comprising
a. providing two therapeutically active agents in effective amounts, one therapeutically active agent being a carboxylic acid, or a salt or an ester thereof; and another therapeutically active agent being a peroxide;
b. providing a multiplicity of solid particles comprising one of the two therapeutically active agents in a manner that substantially excludes contact between the two therapeutically active agents;
c. providing an aqueous liquid wherein said solid particles are dispersed said aqueous liquid phase;
wherein said composition is useful for the treatment of acne.

20. The method of claim 19 wherein said carboxylic acid, or salt or ester thereof comprises azelaic acid, or a salt or ester thereof.

21. The method of claim 19 wherein said carboxylic acid, or salt or ester thereof comprises salicylic acid, or a salt or an ester thereof.

22. The method of claim 19 wherein said carboxylic acid, or salt or ester thereof comprises azelaic acid.

23. The method of claim 19 wherein said peroxide is hydrogen peroxide.

24. The method of claim 19 wherein said peroxide is benzoyl peroxide.

25. A method of stabilizing a carboxylic acid, or a salt or ester thereof in a pharmaceutical product also comprising a peroxide, wherein said product is useful for the treatment of acne, comprising
a. entrapping the carboxylic acid, or salt or ester thereof in a multiplicity of solid particles such that the peroxide is effectively excluded from said particles; or
b. entrapping the peroxide in a number of solid particles such that the carboxylic acid, or salt or ester thereof is effectively excluded from said particles.

26. The method of claim 25 wherein said carboxylic acid, or salt or ester thereof comprises azelaic acid.

27. The method of claim 25 wherein said peroxide is benzoyl peroxide.

28. A pharmaceutical product comprising water, a multiplicity of solid particles, two therapeutically active agents, and a package wherein
a. the first therapeutically active agent is a carboxylic acid, or a salt or an ester thereof; and
b. the second therapeutically active agent is a peroxide; and
c. one of the therapeutically active agents is entrapped in said multiplicity of solid particles such that contact between the therapeutically active agents is substantially reduced; and
d. the package is suitable for dispensing the therapeutically active agents and the product is indicated for the treatment of acne.

29. The product of claim 28 wherein said package has an opening for dispensing the product wherein no particle of said multiplicity of particles is too large to fit through said package opening.

30. The product of claim 29 wherein said package has an opening for dispensing the product which has a diameter greater than 1 cm.

31. The product of claim 29 wherein said opening for dispensing the product has a diameter from 0.5 mm to 1 cm.

32. The product of claim 29 wherein said opening for dispensing the product has a diameter from 1 cm to 10 cm.

33. The composition of claim 1 which further comprises a viscosity enhancing agent.

34. The composition of claim 1 wherein the carboxylic acid, or salt or ester thereof comprises tazarotene.

35. The product of claim 29 wherein the carboxylic acid, or salt or ester thereof comprises tazarotene.

36. The composition of claim 1 wherein the carboxylic acid, or salt or ester thereof is not a retinoid.

37. The product of claim 29 wherein the multiplicity of solid particles have a melting point from 31° C. to 36° C.

38. The method of claim 25 wherein the carboxylic acid, or salt or ester thereof comprises tazarotene.
